(19) United States
(12) Patent Application Publication
(10) Pub. No.: US 2020/0337916 A1
(43) Pub. Date: Oct. 29, 2020

(54) ABSORBENT PAD

(71) Applicant: Gafoor Sheak Abdul SADAT, Kowloon (HK)

(72) Inventor: Gafoor Sheak Abdul SADAT, Kowloon (HK)

(21) Appl. No.: 16/857,806

(22) Filed: Apr. 24, 2020

Related U.S. Application Data
(60) Provisional application No. 62/837,863, filed on Apr. 24, 2019.

(51) Int. Cl. A61F 13/537; A61F 13/511; A61F 13/514

(52) U.S. Cl. CPC A61F 13/53756; A61F 2013/530481; A61F 13/51458; A61F 13/51113

(57) ABSTRACT

The present invention relates to single-use hygiene care product, in particular an absorbent pad, consisting of a liquid absorbent layer in contact with human body and a back layer opposite to the liquid absorbent layer, the back layer being a breathable back sheet; a super absorbent layer arranged between the liquid absorbent layer and the back layer, a lotion layer arranged between the liquid absorbent layer and the super absorbent layer, the lotion layer containing skin-moisturizing lotion. The absorbent pad of the present invention can prevent patients from growing pressure sore due to the peel-off of their dry skin, hence preventing the development of pressure sore and perennial dermatitis. The use of breathable water-proof back layer can ensure air flow and at the same time prevent liquid leakage. The absorbent package is convenient and can effectively protect patients from erythra and perennial dermatitis.

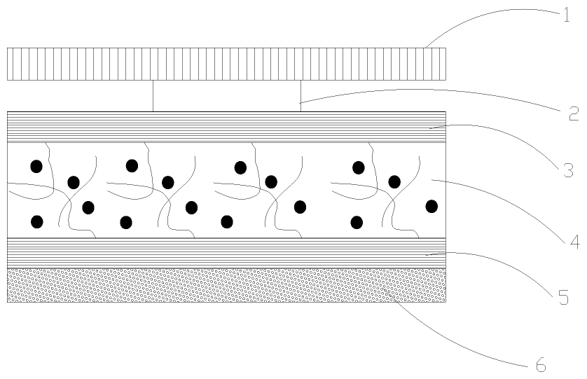

### CROSS REFERENCE TO RELATED APPLICATIONS

This application claims priority of U.S. Provisional Application No. 62/837,863 filed on Apr. 24, 2019 under 35 U.S.C. § 119(e), the entire contents of all of which are hereby incorporated by reference.

### FIELD OF TECHNOLOGY

The invention relates to single-use hygiene care product, in particular an absorbent pad.

### BACKGROUND TECHNOLOGY

Most existing absorbent pads consist of a liquid permeable top layer, a liquid impermeable bottom layer, and a liquid absorbent layer wrapped by the top layer and the bottom layer. Due to the low absorbency of the liquid absorption layer, liquid can easily gather on the top of these absorbent pads. The surface of their liquid permeable top layer will also remain moisturized and is prone to grow bacteria, hence increasing the chance of erythra and perennial dermatitis growth on the human skin in contact with the absorbent pad. Therefore, there is the necessity to provide an absorbent pad that can help patients alleviate and even avoid perennial dermatitis.

### Content of Invention

An absorbent pad is provided according to one aspect of the present invention, consisting of:

a liquid absorbent layer in contact with human body;

a back layer opposite to the liquid absorbent layer, the back layer being a breathable back sheet;

a super absorbent layer arranged between the liquid absorbent layer and the back layer;

and a lotion layer arranged between the liquid absorbent layer and the super absorbent layer, the said lotion layer containing lotion that moisturizes skin.

The materials of different layers are stuck together with hot melt adhesive.

In some embodiments, the liquid absorbent layer is made with spunlace non-woven material. This layer acts as an acquisition layer which facilitates the liquid to spread horizontally and flow through to the second and third layers immediately to avoid accumulation in the liquid absorbent layer. As the first tissue layer, the liquid absorbent layer covers the original the SAP (super absorbent polymer).

In some embodiments, the back layer is a breathable back sheet containing micro porous film, and is a laminate of hydrophobic PE film with non-woven layer. According to ASTM D882-10 method, the back layer’ has MVTR of 1800-2600 g/(m2·d) and tensile strength of 45N (MD) and 35N (CD). Hence the layer allows vapor molecules and heat to transmit through the film effectively and at the same time prevents any leakage of the water molecules. Thus it further prevents leakage through the absorbent pad.

In some embodiments, a super water-absorbing core is arranged in the super absorbent layer. The super water-absorbing core contains pulp and super absorbent polymer (SAP), wherein the SAP is crosslinked sodium polyacrylic acid, CAS number of 9003-04-7, with a maximum overall absorbency of 1600-1800 g (according to ISO 11948-1:1996). With strong absorbency, the SAP can help maintain a balanced pH value for the skin. Even when excrement or excessive leakage occurs, it can prevent the skin from getting over moisturized.

In some embodiments, the lotion layer has a size of 6 inch×30 inch and contains DermPlus+TM lotion. The layer is of color so as to help the patient locate it. The lotion provides a smooth lining which is activated by the body heat when the patient lays on top of it. Hence the layer nourishes the skin and prevents the development of pressure sore caused by the shearing of skin. At the same time, it helps maintain the skin's natural barrier properties so as to prevent perennial dermatitis.

The ingredients of the DermPlus+TM Lotion include Water (Aqua), Glycerin, Helianthus Annuus (Sunflower) Seed Oil, Stearic Acid, Caprylic/Capric Triglyceride, C14-22 Alcohols, Olea Europaea (Olive) Fruit Oil, ButyrospermumParkii (Shea Butter), SimmondsiaChinensis (Jojoba) Seed Oil, Limnanthes Alba (Meadowfoam) Seed Oil, C12-20 Alkyl Glucoside, Algae Extract, Sodium Hyaluronate, Tocopherol, L-Arginine, Glyceryl Caprylate, Zinc Oxide (CI 77947), Sodium Phytate, Fragrance (Parfum), Glyceryl Undecylenate, Citronellol, Coumarin, Hexyl Cinnamic Aldehyde, Isoeugenol, Lilial, D-Limonene, Linalool, Lyral, Alpha Methyl Ionone.

The Jojoba Natural Oil has a very high percentage of iodine which gives its power to heal and fight against harmful bacteria. The Sunflower Seed Oil is rich in essential fatty-acids and antioxidants such as Vitamin E that help maintain healthy skin barrier and decrease transepidermal water loss. The Shea Butter is a skin superfood. It is packed with naturally rich vitamins A, E and F and it provides essential nutrients such as oleic, steraic and palmitic for collagen production.

The DermPlus+TM lotion is hydrophobic and thus cannot transmit through the first tissue carrier. But under the extrusion force of gravity, it can transmit through the liquid absorbent layer in contact with the human body and reach the skin.

In some embodiments, a first tissue carrier is arranged between the lotion layer and the super absorbent layer, and a second tissue carrier is arranged between the super absorbent layer and the back layer. Both the first tissue carrier and the second carrier have a maximum size of 26 inch×30 inch, both carrying water-absorbing polymer material and pulp, with the water-absorbing polymer material being salts and polyacrylamide. Hence both the first tissue carrier and the second carrier carry water-absorbing materials that help absorb liquid. The first tissue carrier the liquid absorbent layer covers the original the SAP (super absorbent polymer). The second tissue carrier is for preventing excessive liquid from leaking through the absorbent pad.

The absorbent pad of the present invention is designed for cover surfaces such as hospital bed and other sitting or lying surfaces of medical setting. It can keep the surface of hospital bed or other medical setting clean, and help patients avoid or alleviate erythra or perennial dermatitis development caused by their prolonged lying due to severe injury or recovery from surgery.

With a lotion layer, the absorbent pad of the present invention can moisturize patients' skin and avoid pressure sores growth caused by the peeling off of dry skin, hence preventing perennial dermatitis. Further, the absorbent pad has a super absorbent layer with a first tissue carrier and a second tissue carrier containing water-absorbing material arranged in its upper and lower layers respectively. It guarantees efficient absorbency and keeps the liquid absorbent layer in low moisture status when being in contact with human skin. In addition, the use of a breathable water-proof back layer can guarantee air flow and prevent leakage through the pad. The absorbent pad of the present invention can act as a incontinence pad and has moisturizing function with lotion. With the two functions combined, the absorbent pad is convenient and can effectively protect patients from erythra and perennial dermatitis.

### FIGURE

The sole FIGURE illustrates the structure of the absorbent pad under one embodiment of the present invention.

### EMBODIMENT

The following is a detailed description of the present invention according to the FIGURE.

The sole FIGURE illustrates the structure of the absorbent pad according to one embodiment of the present invention. As the FIGURE presents, the sheet in contact with the human body is the top sheet, and from the top sheet to the bottom sheet the absorbent pad includes: a liquid absorbent layer (1) in contact with the human body, a lotion layer (2), a first tissue carrier (3), a super absorbent layer (4), a second tissue carrier (5) and a back layer (6). The layers are stuck together with hot melt adhesive, wherein:

The liquid absorbent layer (1) is made with spunlace non-woven material. It acts as an acquisition layer which facilitates the liquid to spread horizontally and flow through to the next layer so as to avoid accumulation in the liquid absorbent layer.

The lotion layer 2, with a size of 6 inch×30 inch, locates in the center of the absorbent pad and contains 6 g of DermPlus+TM lotion. It is of color so as to facilitate the patients to locate it.

The DermPlus+TM lotion is hydrophobic and thus cannot transmit through the first tissue carrier. But under the extrusion force of gravity, it can transmit through the liquid absorbent layer in contact with the human body and reach the skin.

The ingredients of the DermPlus+TM Lotion include Water (Aqua), Glycerin, Helianthus Annuus (Sunflower) Seed Oil, Stearic Acid, Caprylic/Capric Triglyceride, C14-22 Alcohols, Olea Europaea (Olive) Fruit Oil, ButyrospermumParkii (Shea Butter), SimmondsiaChinensis (Jojoba) Seed Oil, Limnanthes Alba (Meadowfoam) Seed Oil, C12-20 Alkyl Glucoside, Algae Extract, Sodium Hyaluronate, Tocopherol, L-Arginine, Glyceryl Caprylate, Zinc Oxide (CI 77947), Sodium Phytate, Fragrance (Parfum), Glyceryl Undecylenate, Citronellol, Coumarin, Hexyl Cinnamic Aldehyde, Isoeugenol, Lilial, D-Limonene, Linalool, Lyral, Alpha Methyl Ionone.

The first tissue carrier (3) has a size of 26 inch×30 inch and carries water-absorbing polymer material and pulp. In the present embodiment, the water-absorbing polymer material is carboxymethyl cellulose.

The super absorbent layer (4) is arranged with a super water-absorbing core inside, and the super water-absorbing core contains pulp and SAP. The SAP used in the present embodiment is crosslinked sodium polyacrylic acid, CAS no. 9003-04-7 and has a maximum absorbency of 1600-1800 g according to ISO 11948-1:1996.

The second tissue carrier (5) has a size of 26 inch×30 inch and carries SAP material and pulp. In the present embodiment, the water-absorbing polymer material is carboxymethyl cellulose.

The back layer (6) is a breathable back sheet containing micro porous film, and is a laminate of lyophobic PE film with non-woven fabric. According to ASTM D882-10, the layer has MVTR of 1800-2600 g/(m2·d) and tensile strength of 45N (MD) and 35N (CD).

In other embodiments, the water-absorbing polymer material in the first tissue carrier (3) and the second tissue carrier (5) can also be polyethylene oxide or polyacrylamide.

The above is only part of the embodiments of the present invention. Any changes and improvements made by people skilled in the art without deviating from the design idea of the present invention all belong to the protection scope of present invention.

1. An absorbent pad, consisting of:
a liquid absorbent layer in contact with human body;
a back layer opposite to the liquid absorbent layer, the back layer being a breathable back sheet;
a super absorbent layer arranged between the liquid absorbent layer and the back layer; and
a lotion layer arranged between the liquid absorbent layer and the super absorbent layer, the said lotion layer containing lotion that moisturizes skin.

2. The absorbent pad according to claim 1, wherein a first tissue carrier is arranged between the said lotion layer and the said super absorbent layer, the said first tissue carrier containing water-absorbing polymer material and pulp.

3. The absorbent pad according to claim 1, wherein a second tissue carrier is arranged between the said super absorbent layer and the back layer, the said second tissue carrier containing water-absorbing polymer material and pulp.

4. The absorbent pad according to claim 2, wherein the water-absorbing polymer material is at least one among salts and polyacrylamide.

5. The absorbent pad according to claim 1, wherein a super water-absorbing core is arranged in the said super absorbent layer.

6. The absorbent pad according to claim 5, wherein the said water-absorbing core contains pulp and super absorbent polymer (SAP).

7. The absorbent pad according to claim 6, wherein the said SAP is crosslinked sodium polyacrylic acid.

8. The absorbent pad according to claim 5, wherein the said back layer is made with a high polymer material laminated with non-woven fabric and PE film, with MVTR of 1800-2600 g/(m2·d) and tensile strength of 45N (MD) and 35N (CD).

9. The absorbent pad according to claim 3, wherein the water-absorbing polymer material is at least one among salts and polyacrylamide.
